(19) United States
(12) Patent Application Publication
(10) Pub. No.: US 2025/0331910 A1
(43) Pub. Date: Oct. 30, 2025

(54) Peltier Cryogenic Treatment System

(71) Applicant: L'Oreal, Paris (FR)

(72) Inventor: Rafael FELICIANO, New Providence, NJ (US)

(73) Assignee: L'Oreal, Paris (FR)

(21) Appl. No.: 18/646,173

(22) Filed: Apr. 25, 2024

(51) Int. Cl. A61B 18/02; A61B 18/00

(52) U.S. Cl. CPC A61B 18/0218; A61B 2018/0047; A61B 2018/00815; A61B 2018/00821

(57) ABSTRACT

A cryogenic device includes a container. The container includes a fluid suitable to be used in skin cryotherapy. The cryogenic device includes a heat exchanger connected to an outlet of the container, wherein the fluid is cooled in the heat exchanger. The cryogenic device includes a multi-stage thermoelectric cooler connected to the heat exchanger to cool the fluid in the heat exchanger. The cryogenic device includes a nozzle after the heat exchanger that discharges the cooled fluid onto a skin surface. The cryogenic device includes a computer configured to open the nozzle when a temperature is reached.

### SUMMARY

A cryogenic device includes a container. The container includes a fluid suitable to be used in skin cryotherapy. The cryogenic device includes a heat exchanger connected to an outlet of the container, wherein the fluid is cooled in the heat exchanger. The cryogenic device includes a multi-stage thermoelectric cooler connected to the heat exchanger to cool the fluid in the heat exchanger. The cryogenic device includes a nozzle after the heat exchanger that discharges the cooled fluid onto a skin surface. The cryogenic device includes a computer configured to open the nozzle when a temperature is reached.

In an embodiment, a reusable carbon dioxide cannister is plugged into a cooling coil system.

In an embodiment, a multi-stage thermoelectric cooler is =attached directly to the cooling coil.

In an embodiment, a temperature sensor is also attached to the cooling coil to measure temperature.

In an embodiment, a computer activates the thermoelectric cooler to super cool the gas contained in the cooling coils. The computer actively monitors the temperature of the coiling coil until the temperature reaches a predetermined temperature.

When the predetermined temperature is reached, the computer activates the solenoid to release the super cooled gas directly onto the skin.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic illustration of a cryogenic device;

FIG. 2 is a schematic illustration of a coil heat exchanger; and

FIG. 3 is a schematic illustration of plate heat exchanger.

### DETAILED DESCRIPTION

Cryotherapy is a treatment method that uses a fluid to freeze lesions on the skin surface. The freezing of the tissue at the surface can destroy the lesions and may cause regeneration of healthier tissue. However, current cryogenic devices use a pressurized cannister of R-152A gas (1,1-difluoroethane). The device can have limitations dispensing in a downward position. Here, downward can mean toward the earth's center. In addition, R-152A needs to be cryogenic in liquid form, and R-152A gas is regulated in several countries.

Accordingly, the present disclosure is meant to address the disadvantages in current devices. The present disclosure can have advantages including, using a safer and cheaper gas for cryogenic application, providing a re-usable or re-chargeable system, controlling the cooling temperature based on the application, and dispensing cryonic gas in any direction.

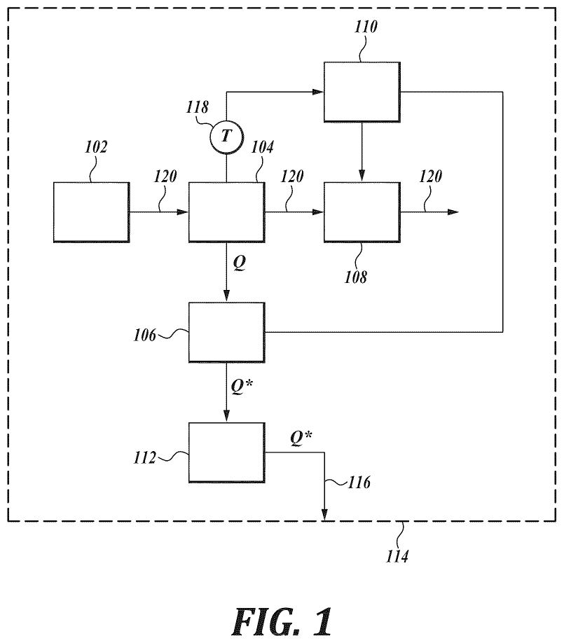

FIG. 1 is a schematic illustration of a cryogenic device 100 according to the disclosure. The cryogenic device 100 includes a container 102. The container 102 includes a fluid 120 suitable to be used in skin cryotherapy. The cryogenic device 100 includes a heat exchanger 104 connected to an outlet of the container 102, wherein the fluid 120 is cooled in the heat exchanger 104. The cryogenic device 100 includes a multi-stage thermoelectric cooler 106 connected to the heat exchanger 104 to cool the fluid 120 in the heat exchanger 104. The cryogenic device 100 includes a nozzle 108 after the heat exchanger 104 that discharges the cooled fluid 120 onto a skin surface. The cryogenic device 100 includes a computer 110 configured to open the nozzle 108 when a temperature is reached to dispense the fluid 120.

The fluid 120 is a gas or a liquid selected from carbon dioxide, nitrogen, nitrous oxide, oxygen, a hydrocarbon such as ether and propane, a hydrochlorofluorocarbon, hydrofluorocarbon, or any combination thereof.

The cryogenic device 100 includes a temperature sensor 118 measuring a temperature at the heat exchanger 104. The temperature sensor 118 can be placed in exterior contact with the heat exchanger 104, such that the temperature sensor 118 can indicate a close approximation of the temperature of the fluid 102. In an embodiment, the temperature sensor 118 can penetrate the heat exchanger wall to directly contact the fluid 120 and measure the fluid temperature. The temperature sensor 118 is a thermocouple, thermistor, and the like. More than one temperature sensor 118 can be used.

The computer 110 is configured to open the nozzle 108 when the temperature is from −40° C. to 0° C. The computer 110 is configured to open the nozzle 108 when the temperature is from −30° C. to −10° C. The nozzle 108 includes a solenoid valve to open and close the flow of fluid 120 out of the nozzle 108. The temperature at which the solenoid valve is permitted to open can be dependent on the type of fluid and/or the particular application of the device 100. For example, the type of lesion can determine the temperature value permitted to open the solenoid valve. Lesions include, but are not limited to, hyperpigmentation, skin tags, viral warts, actinic keratosis, and the like.

The computer 110 includes a power supply. The computer 110, the solenoid within the nozzle 108, and the thermoelectric cooler 106 can be powered by a direct current rechargeable battery, or via an AC to DC converter when plugged into a wall outlet. The computer 110 controls the flow of current to the solenoid in the nozzle 108 and to the multi-stage thermoelectric cooler 106.

The cryogenic device 100 further includes a heat sink 112 connected to the multi-stage thermoelectric cooler 106. Heat sinks 112 can include the use of highly thermally conductive metals, such as copper and aluminum, arranged to increase surface area for heat dissipation. Heat sinks 112 can include the use of liquids, such as water, to remove heat from the multi-stage thermoelectric cooler 106. The heat sink 112 can include the use of a fan. The heat sink 112 removes the heat “Q*” from the multi-stage thermoelectric cooler 106 which increases the efficiency of the multi-stage thermoelectric cooler 106.

In one embodiment, the container 102 is a refillable cartridge. For example, the refillable cartridge is configured to hold from 12 g to 16 g of carbon dioxide.

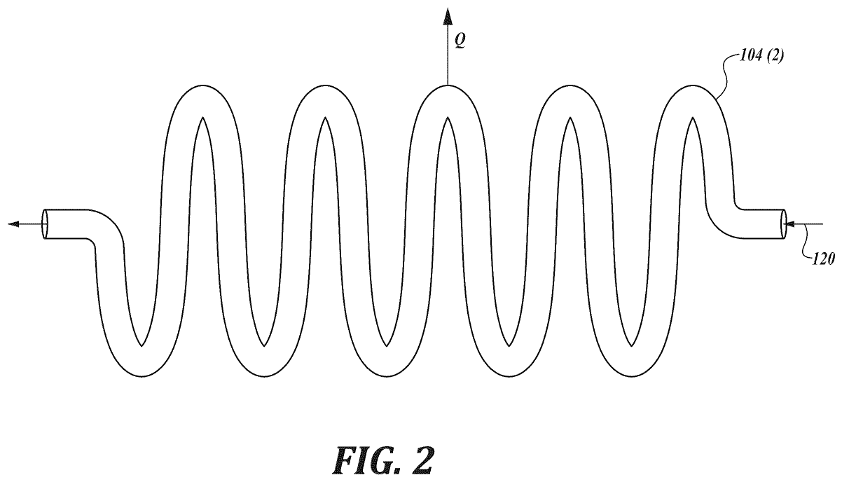

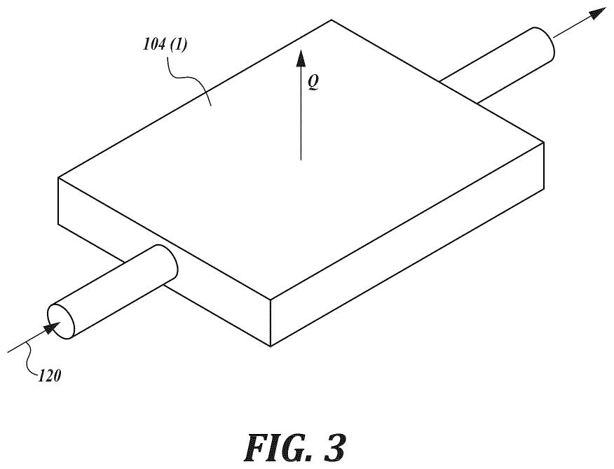

In one embodiment, the heat exchanger 104 includes a coil heat exchanger 104(2) as schematically illustrated in FIG. 2. In one embodiment, the heat exchanger 104 is a plate heat exchanger 104(1) as illustrated in FIG. 3.

The cryogenic device 100 includes one or more housing 114 enclosing the container 102, the heat exchanger 104, the multi-stage thermoelectric cooler 106, the nozzle 108, and the computer 110.

In an embodiment, the one or more housing 114 can be a heat sink for the multi-stage thermoelectric cooler 108. The housing can include highly thermally conductive metal parts, for example. The housing 114 serving as a heat sink is illustrated in FIG. 1 by the line 116 showing the heat Q* is flowing into the one or more housing 114.

In an embodiment, the cryogenic device 100 can be configured to be a handheld device. For example, the cryogenic device 100 includes a battery to power the system, which allows the cryogenic device 100 to be mobile and not limited to a power outlet. The one or more housing 114 can also be configured with a handle to allow maneuvering the device 100 with one hand. However, in an embodiment, the cryogenic device 100 can include a desktop unit connected to a wand unit, in which the wand unit includes the nozzle, and the remaining components can be housed in the desktop unit.

The multi-stage thermoelectric cooler 106 is based on the Peltier effect which causes a difference in temperature when a DC voltage is applied between two semiconductor electrodes. The Peltier effect is achieved using materials such as bismuth and tellurium. The multi-stage thermoelectric cooler 106 used in the disclosure includes one stage or multiple stages to achieve the low temperatures required for cryotherapy. The construction of each stage includes a plurality of “pillars” of alternating P- and N-type semiconductors arranged into pairs. Each of the pillars are packaged between two thermally conductive plates. The plates can be made of ceramics. The pillars are electrically connected in series, such that when a direct current is applied to the terminals of the cooler, heat “Q” is drawn from the cold plate and transferred to the opposite hot plate. The heat has to be removed from the thermoelectric cooler, therefore the thermoelectric cooler needs to be connected to a heat sink that will further remove the heat “Q*” from the hot plate and transfer to the heat sink 112 or the housing 114. The final low temperature achieved by the multi-stage thermoelectric cooler 106 will depend on factors, such as the number of pillars, the surface area of the cold and hot ceramic plates, the efficiency of the heat sink, and other factors.

The computer 110 includes circuitry in order to implement treatment protocols, operably couple two or more components, generate information, determine operation conditions, control the device, and the like. The computer 110 receives a temperature signal 118 from the heat exchanger 104 indicating the temperature of the cold side of the heat exchanger 104 or of the fluid 120. The computer 110 compares the temperature to one or more values stored in its memory. When the computer 110 determines that the temperature meets or exceeds the predetermined temperature value, the computer 110 may allow dispensing the fluid 120. The final determination of dispensing can be made by the user of the device, such as by activating a manual button on the device that opens the solenoid valve.

Circuitry of any type can be used for the computer 110. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof. In an embodiment, circuitry includes one or more ASICs having a plurality of predefined logic components. In an embodiment, circuitry includes one or more FPGA having a plurality of programmable logic components.

In an embodiment, circuitry includes one or more memory devices that, for example, store instructions or data. Non-limiting examples of one or more memory devices include volatile memory (e.g., Random Access Memory (RAM), Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like), persistent memory, or the like. Further non-limiting examples of one or more memory devices include Erasable Programmable Read-Only Memory (EPROM), flash memory, or the like. The one or more memory devices can be coupled to, for example, one or more computing devices by one or more instructions, data, or power buses.

In an embodiment, the device includes circuitry having one or more modules optionally operable for communication with one or more input/output components that are configured to relay user output and/or input. In an embodiment, a module includes one or more instances of electrical, electromechanical, software-implemented, firmware-implemented, or other control devices. Such devices include one or more instances of memory; computing devices; antennas; power or other supplies; logic modules or other signaling modules; gauges or other such active or passive detection components; piezoelectric transducers, shape memory elements, micro-electro-mechanical system (MEMS) elements, or other actuators.

In an embodiment, circuitry includes hardware circuit implementations (e.g., implementations in analog circuitry, implementations in digital circuitry, and the like, and combinations thereof).

In an embodiment, circuitry includes combinations of circuits and computer program products having software or firmware instructions stored on one or more computer readable memories that work together to cause a device 100 to perform one or more methodologies or technologies described herein.

In an embodiment, circuitry includes circuits, such as, for example, microprocessors or portions of microprocessor, that require software, firmware, and the like for operation.

In an embodiment, circuitry includes an implementation comprising one or more processors or portions thereof and accompanying software, firmware, hardware, and the like.

In an embodiment, circuitry includes a baseband integrated circuit or applications processor integrated circuit or a similar integrated circuit in a server, a cellular network device, other network device, or other computing device.

In an embodiment, a cryogenic device 100 comprises a container 102 including a fluid 120 suitable to be used in skin cryotherapy; a heat exchanger 104 connected to an outlet of the container, wherein the fluid is cooled in the heat exchanger; a multi-stage thermoelectric cooler 104 connected to the heat exchanger to cool the fluid in the heat exchanger; a nozzle 108 after the heat exchanger that discharges the cooled fluid; and a computer 110 configured to open the nozzle when a temperature is reached.

The fluid 120 is a gas selected from carbon dioxide, nitrogen, nitrous oxide, oxygen, a hydrocarbon, a hydrofluorocarbon, a hydrochlorofluorocarbon, or any combination thereof.

The cryogenic device 100 can include a temperature sensor 118 measuring a temperature at the heat exchanger 104.

The computer 110 is configured to open the nozzle 108 when the temperature is from −40° C. to 0° C.

The computer 110 is configured to open the nozzle 108 when the temperature is from −30° C. to −10° C.

The cryogenic device 100 can include a heat sink 112 or 114 connected to the multi-stage thermoelectric cooler 106.

The container 102 is a refillable cartridge.

The refillable cartridge is configured to hold from 12 g to 16 g of carbon dioxide.

The heat exchanger 104 includes a coil heat exchanger.

The heat exchanger 104 includes a plate heat exchanger.

The cryogenic device 100 can include one or more housing parts 114.

The one or more housing 114 can enclose the container 102, the heat exchanger 104, the multi-stage thermoelectric cooler 106, the nozzle 108, and the computer 110.

The one or more housing 114 is a heat sink for the multi-stage thermoelectric cooler 106.

The cryogenic device 100 is configured to be a handheld device.

A method of treating a skin lesion comprises with the device 100, applying the cryogenic fluid 120 onto a skin lesion.

The skin lesion includes hyperpigmentation, skin tag, viral wart, actinic keratosis, and the like.

The method can further comprise dispensing the fluid 120, wherein the device 100 is positioned to dispense the fluid other than downwards from the device.

The method can further comprise re-filling the container with the fluid after the container 120 is emptied.

While illustrative embodiments have been illustrated and described, it will be appreciated that various changes can be made therein without departing from the spirit and scope of the invention.

The embodiments of the invention in which an exclusive property or privilege is claimed are defined as follows:

1. A cryogenic device, comprising:
a container including a fluid suitable to be used in skin cryotherapy;
a heat exchanger connected to an outlet of the container, wherein the fluid is cooled in the heat exchanger;
a multi-stage thermoelectric cooler connected to the heat exchanger to cool the fluid in the heat exchanger;
a nozzle after the heat exchanger that discharges the cooled fluid; and
a computer configured to open the nozzle when a temperature is reached.

2. The cryogenic device of claim 1, wherein the fluid is a gas selected from carbon dioxide, nitrogen, nitrous oxide, oxygen, a hydrocarbon, a hydrofluorocarbon, a hydrochlorofluorocarbon, or any combination thereof.

3. The cryogenic device of claim 1, including a temperature sensor measuring a temperature at the heat exchanger.

4. The cryogenic device of claim 3, wherein the computer is configured to open the nozzle when the temperature is from −40° C. to 0° C.

5. The cryogenic device of claim 3, wherein the computer is configured to open the nozzle when the temperature is from −30° C. to −10° C.

6. The cryogenic device of claim 1, further comprising a heat sink connected to the multi-stage thermoelectric cooler.

7. The cryogenic device of claim 1, wherein the container is a refillable cartridge.

8. The cryogenic device of claim 7, wherein the refillable cartridge is configured to hold from 12 g to 16 g of carbon dioxide.

9. The cryogenic device of claim 1, wherein the heat exchanger includes a coil heat exchanger.

10. The cryogenic device of claim 1, wherein the heat exchanger includes a plate heat exchanger.

11. The cryogenic device of claim 1, further comprising one or more housing parts.

12. The cryogenic device of claim 1, further comprising one or more housing enclosing the container, the heat exchanger, the multi-stage thermoelectric cooler, the nozzle, and the computer.

13. The cryogenic device of claim 11, wherein the one or more housing is a heat sink for the multi-stage thermoelectric cooler.

14. The cryogenic device of claim 11, configured to be a handheld device.

15. A method of treating a skin lesion, comprising:
with the device of claim 1, applying the cryogenic fluid onto a skin lesion.

16. The method of claim 15, wherein the skin lesion includes hyperpigmentation, skin tag, viral wart, actinic keratosis, and the like.

17. The method of claim 15, further comprising dispensing the fluid, wherein the device is positioned to dispense the fluid other than downwards from the device.

18. The method of claim 15, further comprising re-filling the container with the fluid after the container is emptied.
